(19) United States
(12) Patent Application Publication
(10) Pub. No.: US 2026/0256357 A1
(43) Pub. Date: Sep. 3, 2026

(54) SYSTEMS AND METHODS FOR RECORDING AND TRANSFERRING DATA FROM A SUBDERMAL IMPLANT DEVICE TO AN EXTERNAL DEVICE VIA A WIRELESS CONNECTION

(71) Applicant: EPI-MINDER PTY LTD, Victoria (AU)

(72) Inventors: Rohan J. Hoare, Dallas, TX (US); John Heasman, North Turramura (AU); Toby McSweeney, Mount Eliza (AU)

(21) Appl. No.: 19/159,908

(22) PCT Filed: Mar. 20, 2023
(86) PCT No.: PCT/AU2023/050197

§ 371 Date: Aug. 26, 2025

Related U.S. Application Data
(60) Provisional application No. 63/451,270, filed on Mar. 10, 2023.

(51) Int. Cl. A61B 5/00; A61B 5/293; A61B 5/372; A61B 5/384; H04L 43/0823

(52) U.S. Cl. CPC A61B 5/0031; A61B 5/0006; A61B 5/293; A61B 5/372; A61B 5/384; A61B 5/721; A61B 5/7267; H04L 43/0847; A61B 2562/0219

(57) ABSTRACT

Methods and systems implement an implanted device that wirelessly transmits gathered data to an external computing device at determined times. The implant device comprises: an electrode configured to gather electrical activity data associated with a brain of a user; communication circuitry configured to wirelessly communicate with the external computing device; a temporary data storage; a processing device; and a computer-readable media storing machine readable instructions. The machine readable instructions cause the processing device to: gather, using the electrode, the electrical activity data; store, at the temporary data storage, the electrical activity data; determine a packet error rate (PER) for communications between the communication circuitry and the external computing device for a plurality of time periods; determine, based at least on the determined PER, a preferred transmission time period of the plurality of time periods during which to transmit the stored electrical activity data to the external computing device.

### TECHNICAL FIELD

The present disclosure relates to systems and methods for monitoring various types of physiological activity in a subject and transmitting data associated with the monitored activity to a computing device. In particular, the disclosure relates to systems and methods for determining a strength of a connection between an implant device monitoring physiological activity in the subject and a computing device to store, process, and/or analyze the data. The disclosure also relates particularly to methods and systems for determining a preferred time period during which to transmit the data based on various determined connection strengths across various times.

### BACKGROUND OF THE DISCLOSURE

Epilepsy is considered the world's most common serious brain disorder, with an estimated 50 million sufferers worldwide and 2.4 million new cases occurring each year. Epilepsy is a condition of the brain characterized by epileptic seizures that vary from brief and barely detectable seizures to more conspicuous seizures in which a sufferer vigorously shakes. Epileptic seizures are unprovoked, recurrent, and due to unexplained causes.

Diagnosing disorders such as epilepsy can be challenging, especially as diagnosis typically requires detailed study of both clinical observations and electrical and/or other signals in the patient's brain and/or body. Diagnosing epilepsy typically requires detailed study of both clinical observations and electrical and/or other signals in the patient's brain and/or body. Particularly with respect to studying electrical activity in the patient's brain (e.g., using electroencephalography to produce an electroencephalogram (EEG)), such study usually requires the patient to be monitored for some period of time. The monitoring of electrical activity in the brain requires the patient to have a number of electrodes placed on the scalp, each of which electrodes is typically connected to a data acquisition unit that samples the signals continuously (e.g., at a high rate) to record the signals for later analysis. Medical personnel monitor the patient to watch for outward signs of epileptic events and review the recorded electrical activity signals to determine whether an event occurred, whether the event was epileptic in nature and, in some cases, the type of epilepsy and/or region(s) of the brain associated with the event. Because the electrodes are wired to the data acquisition unit, and because medical personnel must monitor the patient for outward clinical signs of epileptic or other events, the patient is typically confined to a small area (e.g., a hospital or clinical monitoring room) during the period of monitoring, which can last anywhere from several hours to several days. Moreover, where the number of electrodes placed on or under the patient's scalp is significant, the size of the corresponding wire bundle coupling the sensors to the data acquisition unit may be significant, which may generally require the patient to remain generally inactive during the period of monitoring, and may prevent the patient from undertaking normal activities that may be related to the onset of symptoms.

Any discussion of documents, acts, materials, devices, articles, or the like which has been included in the present background is not to be taken as an admission that any or all of these matters form part of the prior art base or were common general knowledge in the field relevant to the present disclosure as it existed before the priority date of each claim of this application.

### SUMMARY

While some systems exist for longer-term monitoring of a patient outside of a clinical setting, reliable systems require the electrodes to be implanted at a subdermal level, which results in higher quality signal recordings of the EEG over extended time periods. Such is not possible with scalp-based systems. However, such sub-dermal recording devices require the recorded EEG to be sent wirelessly through the body tissue to the outside. This results in a decreased communication efficiency and increased difficulty for maintaining power management for such a system.

As such, it is desirable to have an efficient method for reducing power consumed by the implant device when communicating wirelessly outside the body. Using a separate device (e.g., an external computing device) improves the power efficiency by moving any analysis and other power-heavy tasks to a device with a more easily maintained power supply. However, communications signals between the implanted device and the external computing device are weakened due at least to the organic matter guaranteed to exist between the devices, and attempting to transmit the data without regard to the weakened signals may inefficiently utilize the power of the implanted device and offset gains made.

Therefore, it is further desirable to have a method for determining time periods during which the signal between the implanted device and the external computing device is strongest and/or indicating to a user when and where to position the external computing device, so as to maximize a communication throughput and avoid unnecessary power drain on the implanted device's power supply. By measuring, calculating, and/or otherwise determining factors that contribute to the strength of the signal (e.g., via a proxy for strength such as packet error rate (PER)) and subsequently determining various preferred time period(s) based on the determined factors and/or strength of the signal, a system according to the embodiments described herein may improve the overall functionality. Similarly, by taking action in embodiments in which immediate data transfer is prioritized or in which a preferred time period cannot be easily found, the system can cause an increase in the signal strength (e.g., by using an intermediate device as a relay and/or by causing the user to move the external computing device closer) and, subsequently, the power efficiency.

### BRIEF DESCRIPTION OF THE FIGURES

FIG. 1 is a block diagram depicting an electrode assembly including a local processing device.

FIG. 2 is a block diagram of an embodiment of a processor device communicatively coupled to a sensor array, such as the electrode assembly of FIG. 1.

FIGS. 3A and 3B show side and top views, respectively, of an example electrode device.

FIGS. 3C through 3E show cross-sectional views of portions of the electrode device of FIGS. 3A and 3B.

FIGS. 3F and 3G show top and side views, respectively, of a distal end portion of the electrode device of FIGS. 3A and 3B.

FIG. 3H illustrates an example implantation location of electrodes of an electrode device.

FIG. 3I illustrates an example implantation location of an electrode device.

FIGS. 4A and 4B illustrate example fields that depict a range of wireless communication for an implant device according to one or more embodiments herein.

FIG. 5 illustrates an example system including an implant device, an external computing device, a cloud network, and an optional intermediate device.

FIG. 6 is a flow chart depicting a method for determining a preferred transmission time period, implemented in an implant device.

FIG. 7 is a flow chart depicting a method for determining a preferred transmission time period, implemented in an external computing device.

### DETAILED DESCRIPTION

Embodiments of the present disclosure relate to the monitoring and subsequent transmitting of electrical activity in body tissue of a subject using an array of sensors disposed on or in the patient's body. Certain embodiments relate, for example, to processor devices configured to gather data via electrode arrays implanted (e.g., as subdermal or subdural implants) in a head of a subject (e.g., to monitor brain activity such as epileptic brain activity) and determine time periods during which to transmit such data to an external computing device. In further embodiments, the sensor arrays according to the present disclosure may be for implanting in a variety of different locations of the body, may sense electrical signals, including those generated by electrochemical sensors, and may cooperate with processing devices in various instances as described herein.

Additional embodiments of the present disclosure relate to improving the quality of a connection between the implanted device(s) and the external computing device. Certain embodiments relate, for example, to utilizing an intermediate device to improve the quality of the connection (i.e., by transmitting to the intermediate device to reduce the number of obstacles and improve a packet error rate). Further embodiments relate to initiating an event to cause the user to move the external computing device closer to the implant device (e.g., by causing the external computing device to receive a call).

Various aspects of the systems and methods are described throughout this specification. Unless otherwise specified, aspects of any embodiment that are compatible with another embodiment described herein are considered as contemplated and disclosed embodiments herein. For example, a feature of a particular embodiment described herein, if that feature would be recognized by a person of ordinary skill in the art to be compatible with the features of a second embodiment described herein, should be considered as a possible feature of the second embodiment. Further, embodiments describing features as optional should be considered as disclosing said embodiments both with and without the optional features, and with various optional features in any combination that, in view of this description, would be recognized by a person of ordinary skill in the art as being compatible.

Throughout the present disclosure, embodiments are described in which various elements are optional—present in some, but not all, embodiments of the system. Where such elements are depicted in the accompanying figures and, specifically, in figures depicting block diagrams, the optional elements are generally depicted in dotted lines to denote their optional nature.

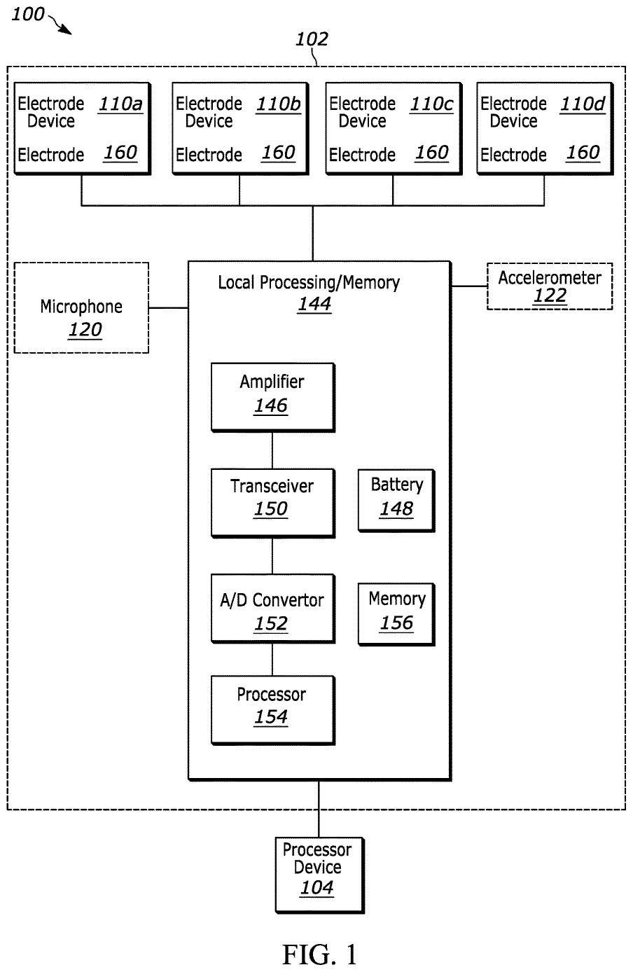

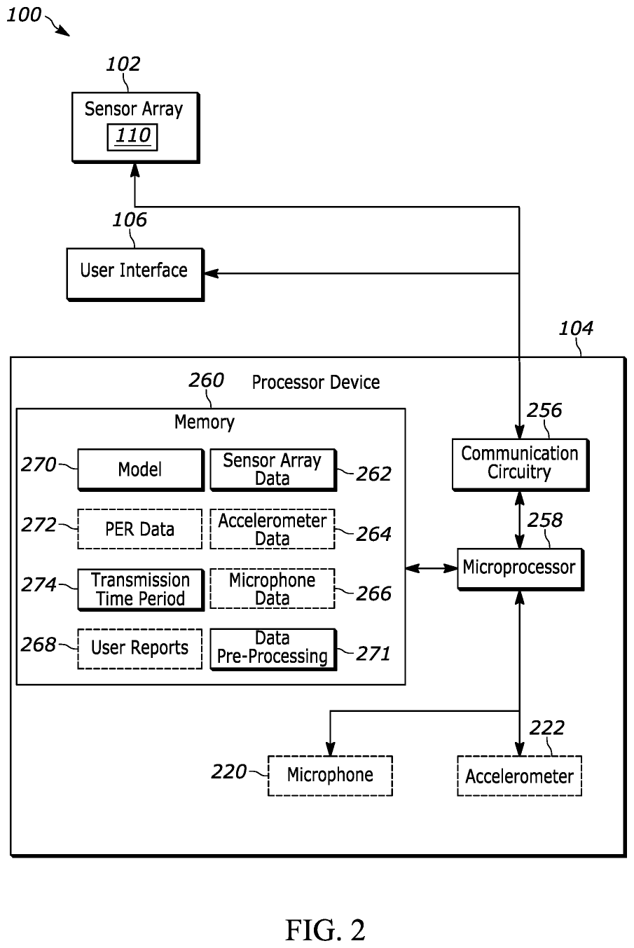

FIG. 1 depicts, in its simplest form, a block diagram of a contemplated system 100 directed to measurement of neurological events and determination of a preferred time for transmission of data (e.g., regarding such events). The system 100 broadly includes a sensor array 102 and a processor device 104. Depending on the embodiment, the system 100 may additionally include a user interface (e.g., user interface 106 as described with regard to FIG. 2 below). The sensor array 102 generally determines preferred times to wirelessly provide data to the processor device 104, which receives the data and uses the data to detect and classify events in the electrical signal data. More particularly, the sensor array 102 may include a local processing/memory device 144 and a plurality of electrode devices 110, each including an electrode 160. The local processing/memory device 144 may include components such as an amplifier 146, a battery 148, a transceiver 150, an analog-to-digital (“A/D”) convertor 152, a processor 154, and/or a memory 156 to generate and transmit data associated with a user's brain, such as EEG and/or PPG data. Depending on the embodiment, the sensor array 102 may additionally include a microphone 120 and/or an accelerometer 122 (or, in some examples, a gyroscope, magnetometer, etc.).

The following description of the sensor array 102 is illustrative in nature. While one of skill in the art would recognize a variety of sensor arrays that may be compatible with the described embodiments, the sensor arrays 102 explicitly described herein may have particular advantages and, in particular, the sensor arrays 102 may include the sensors described in U.S. patent application Ser. No. 16/124,152 (U.S. Patent Application Publication No. 2019/0053730 A1) and U.S. patent application Ser. No. 16/124,148 (U.S. Pat. No. 10,568,574) the specifications of each being hereby incorporated herein by reference, for all purposes.

The local processing device 144 can include a memory 156 to temporarily store signal processing data. In some embodiments, the memory 156 may be of sufficient size to store one to two days of continuous measurement and gathering of data. The local processing device 144 may be similar to a processing device of a type commonly used with cochlear implants, although other configurations are possible. Depending on the embodiment, the transceiver 150 has the capability to transmit data via one or more technologies, such as any of the following techniques, individually or in combination: Wi-Fi, Bluetooth, 5G, WLAN, RFID, and/or any other such technique applicable to the methods described herein.

It will be understood that the instant disclosure contemplates use of transceivers or other components of local processing device 144 in addition to or as a replacement for processor 154 where appropriate according to such techniques. For example, in some embodiments, the device contemplates using the Bluetooth Low Energy (Bluetooth LE) standard (e.g., IEEE 802.15). In such embodiments, the transceiver 150 may be or include one or more transceiver chips, other hardware, and/or software that implements one or more techniques as described herein in addition to or as a replacement for the processor 154. In some such embodiments, a local processing device 144 may therefore measure a packet error rate, for example, using a transceiver 150 according to the Bluetooth LE standard.

The microphone 120 and/or the accelerometer 122 may gather information used by the local processing device 144, other components of the sensor array 102, the processor device 104, and/or another such computing device to determine a preferred transmission time period. For example, the accelerometer 122 gathers information associated with the positioning of the user (e.g., to indicate that the user is horizontal, to indicate that the user is standing or sitting vertically, to indicate that the user is currently walking, etc.). Similarly, the microphone 120 may gather information associated with sounds emitted by the user (e.g., determining that the user is sleeping, determining that the user is speaking, determining that the user is watching a show, etc.). The local processing device 144 may subsequently use the data gathered by the accelerometer 122 and/or microphone 120 to determine a preferred transmission time as described in more detail herein.

The data processed and stored by the local processing device 144 may be raw EEG data or partially processed (e.g., partially or fully compressed) EEG data, for example. The EEG data may be transmitted from the local processing device 144 wirelessly to the processor device 104 for further processing and analyzing of the data at a time determined by the sensor array 102, as described in more detail herein. In one example, the processing device 144 determines a preferred time for transmitting data to the processor device 104 based on a measured packet error rate (PER) and/or factors contributing to the measured PER. In another example, the processor device 104 and/or an external device communicatively coupled to the processor device 104 determines the preferred time based on the measured PER and transmits an indication of the preferred time to the processing device 144 and/or another component of the sensor array 102.

In some embodiments, the local processing device 144 measures the PER by determining a ratio of a number of packets received during communications between the processor device 104 and the sensor array 102 compared to the number of packets actually sent. In further embodiments, the processor device 104 measures the PER and transmits an indication of the PER to the local processing device 144 via the transceiver 150. Depending on the embodiment, the local processing device 144 and/or processor device 104 may determine the PER for various full communications performed (e.g., transfers of data), via one or more test transmissions at designated times, using predetermined data received from a cloud network in conjunction with known factors (e.g., known presences of electromagnetic interference, expected non-organic physical objects, known organic physical objects), water, etc. In some embodiments, the local processing device 144 and/or processor device determines the PER in accordance with the wireless communication standard being used (e.g., Bluetooth LE) as described above.

Depending on the embodiment, the local processing device 144 and/or the processor device 104 may determine the PER, factors that contribute to a low PER, and/or preferred transmission time period according to a trained machine learning (ML) or artificial intelligence (Al) algorithm. In some such embodiments, components of the system 100 use gathered data and/or received data to train an algorithm based on past data specific to the particular communications between the sensor array 102 and the processor device 104.

The trained AI model may be created by an adaptive learning component configured to “train” an AI model (e.g., create the trained Al model) to determine a PER for a particular time period, factors that contribute to a particular PER, and/or a preferred transmission time period using as inputs raw or pre-processed (e.g., by the local processing device 144) data from the sensor array 102 and/or processor device 104. As described herein, the adaptive learning component may use a supervised or unsupervised machine learning program or algorithm. The machine learning program or algorithm may employ a neural network, which may be a convolutional neural network (CNN), a deep learning neural network, or a combined learning module or program that learns in two or more features or feature datasets in a particular area of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques. Machine learning may involve identifying and recognizing patterns in existing data (i.e., training data) such as increased or decreased PERs during particular times, days, etc.

The trained AI model may be created and trained based upon example (e.g., “training data”) inputs or data (which may be termed “features” and “labels”) in order to make valid and reliable predictions for new inputs, such as testing level or production level data or inputs. In supervised machine learning, a machine learning program operating on a server, computing device, or other processor(s), may be provided with example inputs (e.g., “features”) and their associated, or observed, outputs (e.g., “labels”) in order for the machine learning program or algorithm to determine or discover rules, relationships, or other machine learning “models” that map such inputs (e.g., “features”) to the outputs (e.g., “labels”), for example, by determining and/or assigning weights or other metrics to the model across its various feature categories. Such rules, relationships, or other models may then be provided subsequent inputs in order for the model, executing on the server, computing device, or other processor(s), to predict, based on the discovered rules, relationships, or model, an expected output.

In unsupervised learning, the server, computing device, or other processor(s), may be required to find its own structure in unlabeled example inputs, where, for example, multiple training iterations are executed by the server, computing device, or other processor(s) to train multiple generations of models until a satisfactory model (e.g., a model that provides sufficient prediction accuracy when given test level or production level data or inputs) is generated. The disclosures herein may use one or both of such supervised or unsupervised machine learning techniques.

The AI model may be trained using data gathered by the sensor array 102 and/or processor device 104 as inputs. In various embodiments, the AI model is trained using PER as an input and the preferred transmission time as an output, factors that contribute to a low PER (e.g., time of day, user positioning, location, etc.) as an input and preferred transmission time as an output, factors that contribute to PER (e.g., time of day, user positioning, location, etc.) as an input and PER as an output, and other such inputs and outputs as described herein. Depending on the embodiment, the AI model is trained on a schedule (e.g., daily, weekly, etc.), opportunistically (e.g., as the sensor array 102 determines transmission time periods and/or receives feedback from the processor device 104), in response to a user indication, etc. In training the AI model, the sensor array 102, processor device 104, and/or other computing device(s) as described herein may gather data responsive to an indication or determination to train the AI model or may use already-gathered information. Depending on the embodiment, the sensor array 102 and/or processor device 104 may test the trained AI model using test packets or may receive feedback based on communications and determinations performed using the trained AI model.

By determining preferred times to transmit data to the processor device 104 according to a measured PER, the sensor array 102 may reduce time spent searching for an external computing device (e.g., when the wireless signal is not strong enough to reach the device) and transmitting data. As such, the sensor array 102 reduces the overall power consumption within the sensor array 102, reducing the necessary size of a battery 148 and/or improving the period in which the sensor array 102 may function without a potentially intrusive procedure to replace the battery 148, replace the sensor array 102, and/or charge the sensor array 102 or, at a minimum, increasing the period that the user may go without recharging the device (if the device is rechargeable).

The processor device 104 may analyze EEG signals (or other electrical signals) to determine if a target event has occurred. Data regarding the event may be generated by the processor device 104 on the basis of the analysis. In one example, the processor device 104 may analyze brain activity signals to determine if a target event such as an epileptic event has occurred and data regarding the epileptic event (e.g., classification of the event) may be generated by the processor device 104 on the basis of the analysis.

While described herein primarily with respect to epilepsy, it will be clear from the description that the systems and methods herein can be used with and applied to other conditions, as well. Similarly, although the instant descriptions primarily refer to EEG data, it will be understood that data being transferred from the implant device to an external computing device may include data gathered from various sensors implanted in the body (e.g., EEG sensors, PPG sensors, magnetoelastic sensors, etc.) and, in embodiments, microphones and/or accelerometers similar to those described herein.

Turning now to FIG. 2, the systems 100 are presented as a block diagram in greater detail. As depicted in FIG. 2, the system 100 includes, in various embodiments, a microphone 220 and an accelerometer 222, in addition to the sensor array 102, the processor device 104, and the user interface 106. Each of the sensor array 102, the microphone 220, and the accelerometer 222 may sense or collect respective data and wirelessly communicate the respective data to the processor device 104 at a determined transmission time period. In further embodiments, the microphone 220 and the accelerometer 222 may be used to determine the transmission time period. As should be understood, in embodiments, the sensor array 102 may include an array of electrode devices 110 that provide electrical signal data and, in particular, provide electrical signal data indicative of brain activity of the patient (e.g., EEG signal data). As should also be understood in view of the description herein, the sensor array 102 may be disposed beneath the scalp of the patient-on and/or extending into the cranium-so as to facilitate accurate sensing of brain activity. However, in embodiments, it is also contemplated that the sensor array 102 need not be placed beneath the scalp, but instead be implanted (e.g., at a subdermal or subdural level) elsewhere on a patient's body. In such embodiments, the sensor array 102 receives other signals from the patient rather than EEG signals (e.g., PPG signals, chemical information, etc.).

In the embodiment of FIG. 2, the sensor array 102 includes components and functionalities as described with regard to FIG. 1 above and is communicatively coupled to the processor device 104 and the user interface 106. The user interface 106 may facilitate self-reporting by the patient of any of various data including events perceived by the patient, as well as medication types, doses, dose times, patient mood, potentially relevant environmental data, and the like. The user interface 106 may also facilitate output of classification results, programming of the unit for a particular patient, calibration of the sensor array 102, etc.

The processor device 104 includes communication circuitry 256, a microprocessor 258, and a memory device 260. In some embodiments, the processor device 104 additionally includes the microphone 220 and/or accelerometer 222, as described above. The microprocessor 258 may be any known microprocessor configurable to execute the routines necessary for determining a PER and/or determining a preferred time for transmitting data, including, by way of example and not limitation, general purpose microprocessors (GPUs), RISC microprocessors, digital signal processors (DSPs), application specific integrated circuits (ASICs), and field-programmable gate arrays (FPGAs).

The communication circuitry 256 may be any transceiver and/or receiver/transmitter pair that facilitates communication with the various devices from which the processor device 104 receives data and/or transmits data. The communication circuitry 256 is communicatively coupled, in a wired or wireless manner, to each of the sensor array 102, the microphone 220, the accelerometer 222, and the user interface 106. Additionally, the communication circuitry 256 is coupled to the microprocessor 258, which, in addition to executing various routines and instructions for performing analysis, may also facilitate storage in the memory 260 of data received, via the communication circuity 256, from the sensor array 102, the microphone 220, the accelerometer 222, and the user interface 106.

The communication circuitry 256 receives communications from the sensor array 102, after which the processor device may determine PER data 272 and/or a preferred transmission time 274 according to a model 270 as described in more detail below. Depending on the embodiment, the communications may be test communications for the purposes of determining PER, determined PER data, full data communications, etc.

The memory 260 may include both volatile memory (e.g., random access memory (RAM)) and non-volatile memory, in the form of either or both of magnetic or solid state media. In addition to an operating system (not shown), the memory 260 may store sensor array data 262 received from the sensor array 102, accelerometer data 264 received from the accelerometer(s) 252, microphone data 266 received from the microphone(s) 250, user report data 268 received from the user (and/or other person such as a caregiver) via the user interface 106, and/or any other such data. In particular, the user report data 268 may include reports from the user, received via the user interface 106, of timing, commands, messages, etc. from a user. For example, depending on the embodiment, the user report data 268 may include indications that data transfer should not occur between certain hours, during certain days, at certain locations, etc.

As is described with regard to FIG. 1 above, the memory 260 may also store a model 270 for determining a PER and/or preferred transmission time period according to communications received via the communication circuitry. The PER data 272 and/or transmission time period 274 output by the model 270 may be stored in the memory 260. In some embodiments, the processor device 104 transmits the data to the sensor array 102 and deletes the PER data 272 and/or transmission time period 274 from the memory 260. A data pre-processing routine 271 may provide pre-processing of the received communications from the communication circuitry 256 prior to analyzing the communications with the model 270. As will be understood, the data pre-processing routine 271 may provide a range of pre-processing steps including, for example, filtering and extraction from the communications of various features and/or factors. Of course, it should be understood that wherever a routine, model, or other element stored in memory is referred to as receiving an input, producing or storing an output, or executing, the routine, model, or other element is, in fact, executing as instructions on the microprocessor 258. Further, those of skill in the art will appreciate that the model or routine or other instructions would be stored in the memory 260 as executable instructions, which instructions the microprocessor 258 would retrieve from the memory 260 and execute. Further, the microprocessor 258 should be understood to retrieve from the memory 260 any data necessary to perform the executed instructions (e.g., data required as an input to the routine or model), and to store in the memory 260 the intermediate results and/or output of any executed instructions.

The microphone 220 and/or the accelerometer 222 may gather information used by the microprocessor 258, other components of the processor device 104, the sensor array 102, and/or another such computing device to determine a preferred transmission time period. For example, the accelerometer 222 gathers information associated with the positioning of the user (e.g., to indicate that the user is horizontal, to indicate that the user is standing or sitting vertically, to indicate that the user is currently walking, etc.). Similarly, the microphone 220 may gather information associated with sounds emitted by the user (e.g., determining that the user is sleeping, determining that the user is speaking, determining that the user is watching a show, etc.).

The processor device 104 receives data from the sensor array 102 and/or the user interface 106 and, using the received data, may detect events of interest and/or determine a current state of the user. Similarly, the processor device 104 may use data gathered by one or more components of the processor device 104 itself (e.g., the microphone 220 and/or accelerometer 222). For example, in some embodiments, the accelerometer 222 or an accelerometer in the sensor array 102 may provide data indicating that a user is laying down and/or sitting for long periods of time (e.g., 1 hour, 6 hours, 8 hours, etc.). The processor device 104 may correlate the accelerometer data with a PER and determine that, for example, the PER is improved when the user is laying down for long periods of time (e.g., because the user has an external computing device such as a cell phone nearby while sleeping). As such, the processor device 104 may determine a PER for periods of time with similar accelerometer data and/or determine a preferred transmission time period based on such accelerometer, microphone, sensor, etc. data.

In some embodiments, the processor device 104 may provide live feedback regarding PER data and/or preferred transmission times. For example, the processor device 104 may generate or use feedback regarding the PER data and/or preferred transmission times and tendency of the user to adhere to recommendations regarding such to gamify the process. As such, the processor device 104 may determine that a user is following recommendations to improve PER, following indications to perform contact events as discussed in more detail herein, responding to alerts and/or messages, etc. The processor device 104 may then offer virtual or real-world rewards to a user based on the overall PER, adherence to preferred transmission times, etc. In some such embodiments, an AI model may determine when a user follows recommendations and/or may be used to generate feedback regarding the PER data, preferred transmission times, etc.

Although FIG. 2 depicts a single external computing device (e.g., processor device 104), it will be understood that various embodiments may include additional or alternative external computing devices, such as one or more caregiver devices and/or one or more physician devices. In such embodiments, the additional external computing devices may receive alerts or alarms from the processor device 104 about occurring or recently occurred events (e.g., seizures or other medical events). In some embodiments, the additional external devices may include an instance of the user interface 106, allowing the caregiver to provide information about the state of the patient.

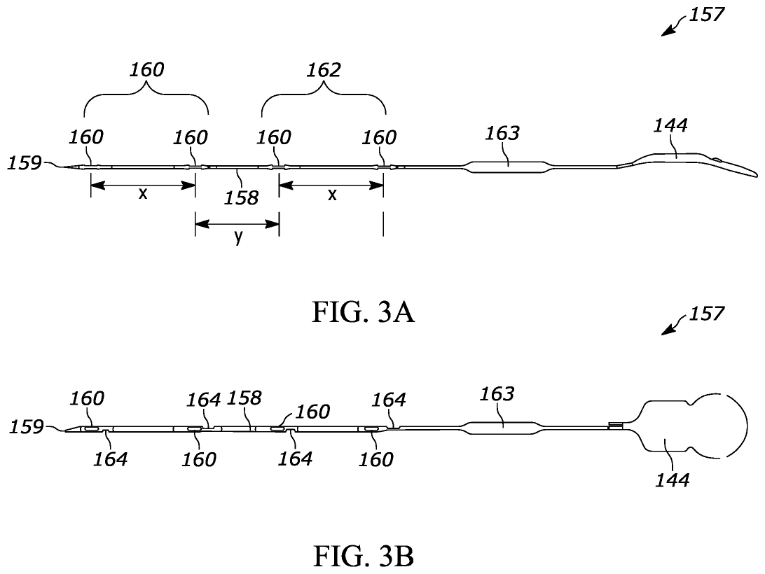

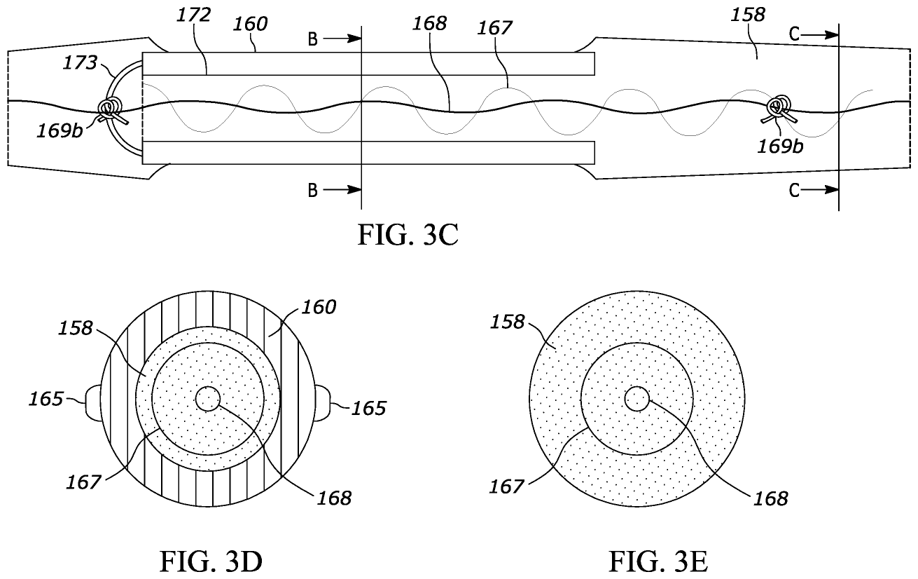

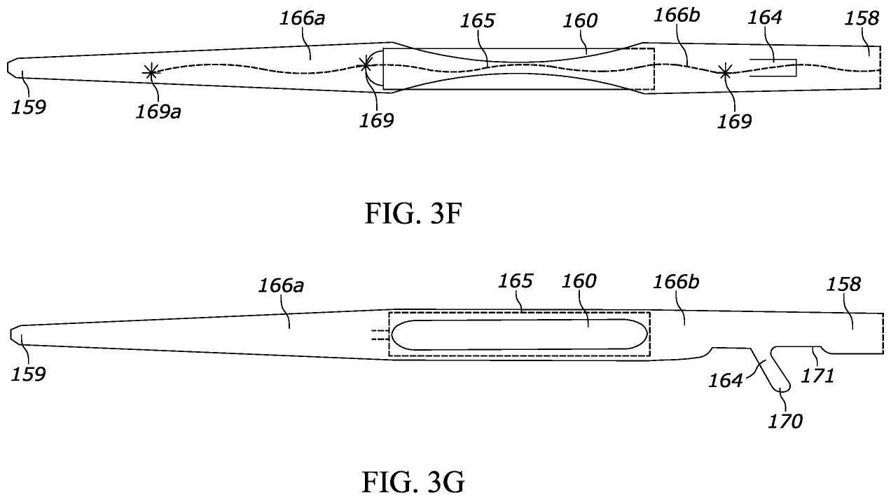

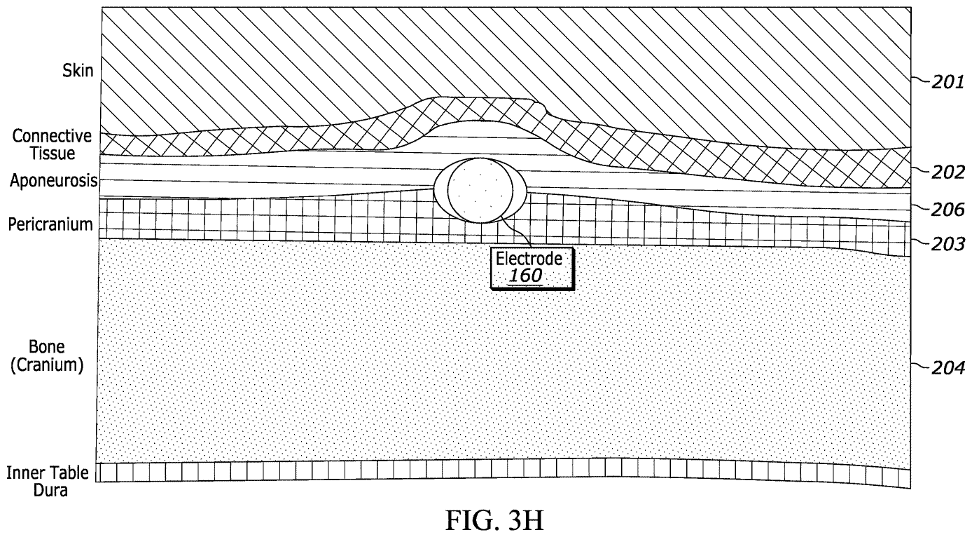

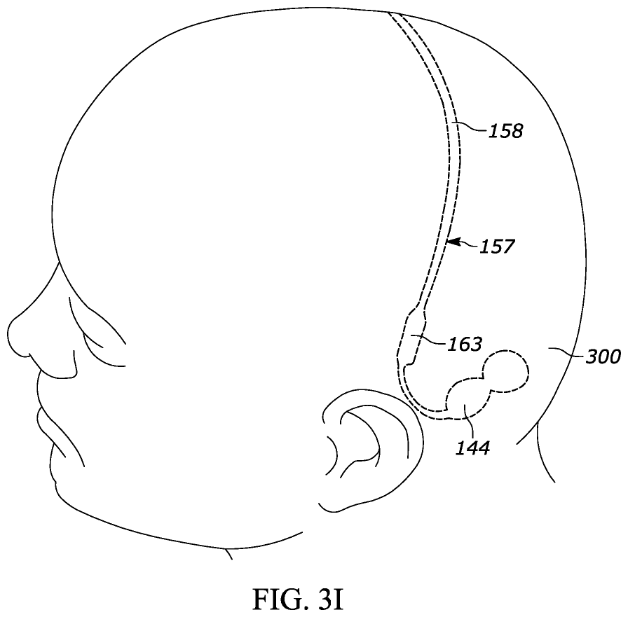

FIGS. 3A through 3I illustrate an embodiment of a sensor array 102, such as that described in U.S. patent application Ser. No. 16/797,315, entitled “Electrode Device for Monitoring and/or Stimulating Activity in a Subject,” the entirety of which is hereby incorporated by reference herein. With reference to FIGS. 3A and 3B, in one embodiment an electrode device 157 is provided comprising an elongate, implantable body 158 and a plurality of electrodes 160 positioned along the implantable body 158 in the length direction of the implantable body 158. At a proximal end of the implantable body 158, a processing unit 144 is provided for processing electrical signals that can be sent to and/or received from the electrodes 160. Though not required, in some embodiments, an electrical amplifier 163 (e.g., a pre-amp) is positioned in the implantable body 158 between the electrodes 160 and the processing unit 144. In an alternative embodiment, the electrical amplifier 163 may be integrated into the processing unit 144 of the electrode device 157, instead of being positioned in the implantable body 158.

With reference to FIG. 3C, which shows a cross-section of a portion of the electrode device 157 adjacent one of the electrodes 160, the electrodes 160 are electrically connected, e.g., to the amplifier 163 and processing unit 144, by an electrical connection 167 that extends through the implantable body 158. A reinforcement device 168 is also provided in the electrode device 157, which reinforcement device 168 extends through the implantable body 158 and limits the degree by which the length of the implantable body 158 can extend under tension.

In this embodiment, referring to FIGS. 3A and 3B, four electrodes 160 are provided that are spaced along the implantable body 158 between the amplifier 163 and a distal tip 159 of the implantable body 158. The distal tip 159 of the implantable body 158 is tapered. The four electrodes 160 are configured into two electrical pairs 161, 162 of electrodes, the two most distal electrodes 160 providing a first pair of electrodes 161 and the two most proximal electrodes 160 providing a second pair of electrodes 162. In this embodiment, the electrodes 160 of the first pair 160 are spaced from each other at a distance x of about 40 to 60 mm, e.g., about 50 mm (measured from center-to-center of the electrodes 160) and the electrodes 160 of the second pair 122 are also spaced from each other at a distance x of about 40 to 60 mm, e.g., about 50 mm (measured from center-to-center of the electrodes 160). The first and second electrode pairs 161, 162 are spaced from each other at a distance y of about 30 to 50 mm, e.g., about 40 mm (measured from center-to-center of the electrodes of the two pairs that are adjacent each other).

With reference to FIGS. 3D and 3E, which provide cross-sectional views along lines B—B and C—C in FIG. 3C, respectively, the implantable body 158 has a round, e.g., substantially circular or ovate, cross-sectional profile. Similarly, each of the electrodes 160 has a round, e.g., substantially circular or ovate, cross-sectional profile. Each of the electrodes 160 extend circumferentially, completely around a portion of the implantable body 158. By configuring the implantable body 158 and electrodes 160 in this manner, the exact orientation of the implantable body 158 and electrodes 160, when implanted in a subject, is less critical. For example, the electrodes 160 may interact electrically with tissue in substantially any direction. In this regard, the electrodes 160 may be considered to have a 360-degree functionality. The round cross-sectional configuration can also provide for easier insertion of the implantable portions of the electrode device 157 to the target location and with less risk of damaging body tissue. For example, the implantable body 158 can be used with insertion cannulas or sleeves and may have no sharp edges that might otherwise cause trauma to tissue.

In this embodiment, the implantable body 158 is formed of an elastomeric material such as medical grade silicone. Each electrode 160 comprises an annular portion of conductive material that extends circumferentially around a portion of the implantable body 158. More specifically, each electrode 160 comprises a hollow cylinder of conductive material that extends circumferentially around a portion of the implantable body 158 and, in particular, a portion of the elastomeric material of the implantable body 158. The electrodes 160 may be considered ‘ring’ electrodes.

Referring back to the embodiment of FIGS. 3A and 3B, and with further reference to FIGS. 3F and 3G, to strengthen the engagement between the electrodes 160 and the implantable body 158, straps 165 are provided in this embodiment that extend across an outer surface of each electrode 160. In this embodiment, two straps 165 are located on substantially opposite sides of each electrode 160 in a direction perpendicular to the direction of elongation of the implantable body 158. The straps 165 are connected between sections 166a, 166b of the implantable body 158 that are located on opposite sides of the electrodes 160 in the direction of elongation of implantable body, which sections 166a, 166b are referred to hereinafter as side sections. The straps 165 can prevent the side sections 166a, 166b from pulling or breaking away from the electrodes 160 when the implantable body 158 is placed under tension and/or is bent. In this embodiment, the straps 165 are formed of the same elastomeric material as the side sections 166a, 166b. The straps 165 are integrally formed with the side sections 166a, 166b. From their connection points with the side sections 166a, 166b, the straps 165 decrease in width towards a central part of each electrode 160, minimizing the degree to which the straps 165 cover the surfaces of the electrodes 160 and ensuring that there remains a relatively large amount of electrode surface that is exposed around the circumference of the electrodes 160 to make electrical contact with adjacent body tissue. With reference to FIG. 3D, around a circumference of each electrode 160, at least 75%, at least 80%, at least 85%, or at least 90% of the outer electrode surface may be exposed for electrical contact with tissue, for example.

In alternative embodiments, a different number of straps 165 may be employed (e.g., one, three, four or more straps 165). Where a greater number of straps 165 is employed, the width of each strap 165 may be reduced. The straps 165 may be distributed evenly around the circumference of each electrode 160 or distributed in an uneven manner. Nevertheless, in some embodiments, the straps 165 may be omitted, ensuring that all of the outer electrode surface is exposed for electrical contact with tissue around a circumference of the electrode 160.

As indicated above, in some embodiments, the implantable body 158 is formed of an elastomeric material such as silicone. The elastomeric material allows the implantable body 158 to bend, flex and stretch such that the implantable body 158 can readily contort as it is routed to a target implantation position and can readily conform to the shape of the body tissue at the target implantation position. The use of elastomeric material also ensures that any risk of trauma to the subject is reduced during implantation or during subsequent use.

In embodiments of the present disclosure, the electrical connection 167 to the electrodes 160 comprises relatively fragile platinum wire conductive elements. With reference to FIGS. 3C to 3E, for example, to reduce the likelihood that the platinum wires will break or snap during bending, flexing and/or stretching of the implantable body 158, the electrical connection 167 is provided with a wave-like shape and, more specifically, a helical shape in this embodiment, although other non-linear shapes may be used. The helical shape, for example, of the electrical connection 167 enables the electrical connection 167 to stretch, flex and bend in conjunction with the implantable body 158. Bending, flexing, and/or stretching of the implantable body 158 typically occurs during implantation of the implantable body 158 in a subject and upon any removal of the implantable body 158 from the subject after use.

As indicated above, a reinforcement device 168 is also provided in the electrode device 157, which reinforcement device 168 extends through the implantable body 158 and is provided to limit the degree by which the length of the implantable body 158 can extend under tension. The reinforcement device 168 can take the bulk of the strain placed on the electrode device 157 when the electrode device 157 is placed under tension. The reinforcement device 168 is provided in this embodiment by a fiber (e.g., strand, filament, cord or string) of material that is flexible, and which has a high tensile strength. In particular, a fiber of ultra-high-molecular-weight polyethylene (UHMwPE), e.g., Dyneema™, is provided as the reinforcement device 168 in the present embodiment. The reinforcement device 168 extends through the implantable body 158 in the length direction of the implantable body 158 and is generally directly encased by the elastomeric material of the implantable body 158.

The reinforcement device 168 may comprise a variety of different materials in addition to or as an alternative to UHMwPE. The reinforcement device may comprise other plastics and/or non-conductive material such as a poly-paraphenylene terephthalamide, e.g., Kevlar™. In some embodiments, a metal fiber or surgical steel may be used.

Similar to the electrical connection 167, the reinforcement device 168 also has a wave-like shape and, more specifically, a helical shape in this embodiment, although other non-linear shapes may be used. The helical shape of the reinforcement device 168 is different from the helical shape of the electrical connection 167. For example, as evident from FIGS. 3C to 3E, the helical shape of the reinforcement device 168 has a smaller diameter than the helical shape of the electrical connection 167. Moreover, the helical shape of the reinforcement device 168 has a greater pitch than the helical shape of the electrical connection 167.

When the implantable body 168 is placed under tension, the elastomeric material of the implantable body will stretch, which in turn causes straightening of the helical shapes of both the electrical connection 167 and the reinforcement device 168. As the electrical connection 167 and the reinforcement device straighten 168, their lengths can be considered to increase in the direction of elongation of the implantable body 158. Thus, the lengths of each of the electrical connection 167 and the reinforcement device 168, in the direction of elongation of the implantable body 158, are extendible when the implantable body 158 is placed under tension.

For each of the electrical connection 167 and the reinforcement device 168, a theoretical maximum length of extension in the direction of elongation of the implantable body 158 is reached when its helical shape (or any other non-linear shape that may be employed) is substantially completely straightened. However, due to the differences in the helical shapes of the electrical connection 167 and the reinforcement device 168, the maximum length of extension of the reinforcement device 168 is shorter than the maximum length of extension of the electrical connection 167. Therefore, when the implantable body 158 is placed under tension, the reinforcement device 168 will reach its maximum length of extension before the electrical connection 167 reaches its maximum length of extension. Indeed, the reinforcement device 168 can make it substantially impossible for the electrical connection 167 to reach a maximum length of extension. Since the electrical connection 167 can be relatively fragile and prone to breaking, particularly when placed under tension, and particularly when the electrical connection 167 reaches a maximum length of extension, the reinforcement device 168 can reduce the likelihood that the electrical connection 167 will be damaged when the implantable body 158 is placed under tension. In contrast to the electrical connection 167, when the reinforcement device 168 reaches its maximum length of extension, a high tensile strength allows the reinforcement device 168 to bear a significant amount of strain placed on the electrode device 157, preventing damage to the electrical connection 167 and other components of the electrode device 157.

In consideration of other components of the electrode device 157 that are protected from damage by the reinforcement device 168, it is notable that the implantable body 158 can be prone to damage or breakage when placed under tension. The elastomeric material of the implantable body 158 has a theoretical maximum length of extension in the direction of elongation when placed under tension, the maximum length of extension being the point at which the elastomeric material reaches its elastic limit. In this embodiment, the maximum length of extension of the reinforcement device 168 is also shorter than the maximum length of extension of the implantable body 158. Thus, when the implantable body 158 is placed under tension, the reinforcement device 168 will reach its maximum length of extension before the implantable body 158 reaches its maximum length of extension. Indeed, the reinforcement device 168 can make it substantially impossible for the implantable body 158 to reach its maximum length of extension. Since elastomeric material of the implantable body 158 can be relatively fragile and prone to breaking, particularly when placed under tension, and particularly when it reaches its elastic limit, the reinforcement device 168 can reduce the likelihood that the implantable body 158 will be damaged when placed under tension.

In this embodiment, the helical shapes of the reinforcement device 168 and the electrical connection 158 are provided in a concentric arrangement. Due to its smaller diameter, the reinforcement device 168 can locate radially inside of the electrical connection 167. In view of this positioning, the reinforcement device 168 provides a form of strengthening core to the implantable body 158. The concentric arrangement can provide for increased strength and robustness while offering optimal surgical handling properties, with relatively low distortion of the implantable body 158 when placed under tension.

As indicated, the reinforcement device 168 is directly encased by the elastomeric material of the implantable body 158. The helically shaped reinforcement device 168 therefore avoids contact with material other than the elastomeric material in this embodiment. The helically shaped reinforcement device is not entwined or intertwined with other strands or fibers, for example (e.g., as opposed to strands of a rope), ensuring that there is a substantial amount of give possible in relation to the helical shape. The helical shape can move to a straightened configuration under tension as a result, for example.

The arrangement of the reinforcement device 168 is such that, when the implantable body 158 is placed under tension, the length of the reinforcement device 168 is extendible by about 20% of its length when the implantable body 158 is not under tension. Nevertheless, in embodiments of the present disclosure, a reinforcement device 168 may be used that is extendible by at least 5%, at least 10%, at least 15%, at least 20%, or at least 25% or otherwise, of the length of the reinforcement device when the implantable body 158 is not under tension. The maximum length of extension of the reinforcement device 168 in the direction of elongation of the implantable body 158 may be about 5%, about 10%, about 15%, about 20%, or about 25% or otherwise of its length when the implantable body 158 is not under tension.

As represented in FIG. 3C, the reinforcement device 168 has a relatively uniform helical configuration along its length. However, in some embodiments, the shape of the reinforcement device 168 can be varied along the length. For example, the reinforcement device 168 can be straighter (e.g., by having a helical shape with smaller radius and/or greater pitch) adjacent the electrodes 160 in comparison to at other portions of the implantable body 158. By providing this variation in the shape of the reinforcement device 168, stretching of the implantable body 158 may be reduced adjacent the electrodes 160, where there could otherwise be a greater risk of the electrodes 160 dislocating from the implantable body 158. This enhanced strain relief adjacent the electrodes 160 can be provided while still maintaining the ability of the reinforcement device 168, and therefore implantable body 158, to stretch to a desirable degree at other portions of the implantable body 158.

As indicated, the electrical connection 167 in this embodiment comprises relatively fragile platinum wire conductive elements. At least 4 platinum wires are provided in the electrical connection 167 to each connect to a respective one of the four electrodes 160. The wires are twisted together and electrically insulated from each other. Connection of a platinum wire of the electrical connection 167 to the most distal of the electrodes 160 is illustrated in FIG. 3C. As can be seen, the wire is connected to an inner surface 172 of the electrode 160, adjacent a distal end of the electrode 160, albeit other connection arrangements can be used.

The reinforcement device 168 extends through the hollow center of each of the electrodes 160. The reinforcement device 168 extends at least from the distal most electrode 160, and optionally from a region adjacent the distal tip 159 of the implantable body 158, to a position adjacent the amplifier 163. In some embodiments, the reinforcement device 168 may also extend between the amplifier 163 and the processing unit 144. In some embodiments, the reinforcement device 168 may extend from the distal tip 159 and/or the distal most electrode 160 of the implantable body 158 to the processing unit 144.

To prevent the reinforcement device 168 from slipping within or tearing from the elastomeric material of the implantable body 158, a series of knots 169 are formed in the reinforcement device 168 along the length of the reinforcement device 168. For example, with reference to FIG. 3F, a knot 169a can be formed at least at the distal end of the reinforcement device 168, adjacent the distal tip 159 of the implantable body 158, and/or knots 169 can be formed adjacent one or both sides of each electrode 160. The knots may alone provide resistance to movement of the reinforcement device 168 relative to the elastic material of the implantable body 158 and/or may be used to fix (tie) the reinforcement device 168 to other features of the device 157.

In the present embodiment for example, as illustrated in FIG. 3C, the reinforcement device 168 is fixed, via a knot 169b, to each electrode 160. To enable the reinforcement device 168 to be fixed to the electrode 160, the electrode 160 comprises an extension portion 173 around which knots 169 of the reinforcement device 168 can be tied. As shown in FIG. 3C, the extension portion 173 can include a loop or arm of material that extends across an open end of the hollow cylinder forming the electrode 160.

With reference to FIGS. 3A, 3B, 3F, and 3G, the electrode device 158 comprises at least one anchor 164, and in this embodiment of plurality of anchors 164. The plurality of anchors 164 are positioned along a length of the implantable body 158, each adjacent a respective one of the electrodes 160. Each anchor 164 is configured to project radially outwardly from the implantable body 158 and specifically, in this embodiment, at an angle towards a proximal end of the implantable body 158. Each anchor 164 is in the form of a flattened appendage or fin with a rounded tip 170. The anchors 164 are designed to provide stabilization to the electrode device 157 when it is in the implantation position.

When implanted, a tissue capsule can form around each anchor 164, securing the anchor 164 and therefore the implantable body 158 into place. In this embodiment, the anchors 164 are between about 0.5 mm and 2 mm in length, e.g., about 1 mm or 1.5 mm in length.

So that the anchors 164 do not impede implantation of the electrode device 157, or removal of the electrode device 157 after use, each anchor 164 is compressible. The anchors 164 are compressible (e.g., foldable) to reduce the degree by which the anchors 164 projects radially outwardly from the implantable body 158. To further reduce the degree by which the anchors 164 project radially outwardly from the implantable body 158 when compressed, a recess 171 is provided in a surface of the implantable body 158 adjacent each anchor 164. The anchor 164 is compressible into the recess 171. In this embodiment, the anchors 164 project from a bottom surface of the respective recess 171 and the recess 171 extends on both proximal and distal sides of the anchor 164. Accordingly, the anchors 164 can be compressed into the respective recesses in either a proximal or distal direction. This has the advantage of allowing the anchors 164 to automatically move into a storage position in the recess 171 when pulled across a tissue surface or a surface of a implantation tool such as delivery device, in either of a proximal and a distal direction.

The electrode device 157 of the present embodiment is configured for use in monitoring electrical activity in the brain and particularly for monitoring electrical activity relating to epileptic events in the brain. The electrode device 157 is configured to be implanted at least partially in a subgaleal space between the scalp and the cranium. At least the electrodes 160 and adjacent portions of the implantable body 158 are located in the subgaleal space.

An illustration of the implantation location of the electrodes 160 is provided in FIG. 3H. As can be seen, the electrodes 160 are located in particular in a pocket between the galea aponeurotica 206 and the pericranium 203. When implanted, the first and second electrode pairs 161, 162 are located on respective sides of the midline of the head of the subject in a substantially symmetrical arrangement. The first and second electrode pairs 161, 162 therefore locate over the right and left hemispheres of the brain, respectively. For example, the first electrode pair 161 can be used to monitor electrical activity at right hemisphere of the brain and the second electrode pair 162 can be used to monitor electrical activity at the left hemisphere of the brain, or vice-versa. Independent electrical activity data may be recorded for each of the right and left hemispheres, e.g., for diagnostic purposes. To position the electrodes pairs 161, 162 over the right and left hemispheres of the brain, the implantable body 158 of the electrode device 157 is implanted in a medial-lateral direction over the cranium of the subject's head. The electrode pairs 161, 162 are positioned away from the subject's eyes and chewing muscles to avoid introduction of signal artifacts from these locations. The electrode device 157 implanted under the scalp in a position generally as illustrated in FIG. 3I.

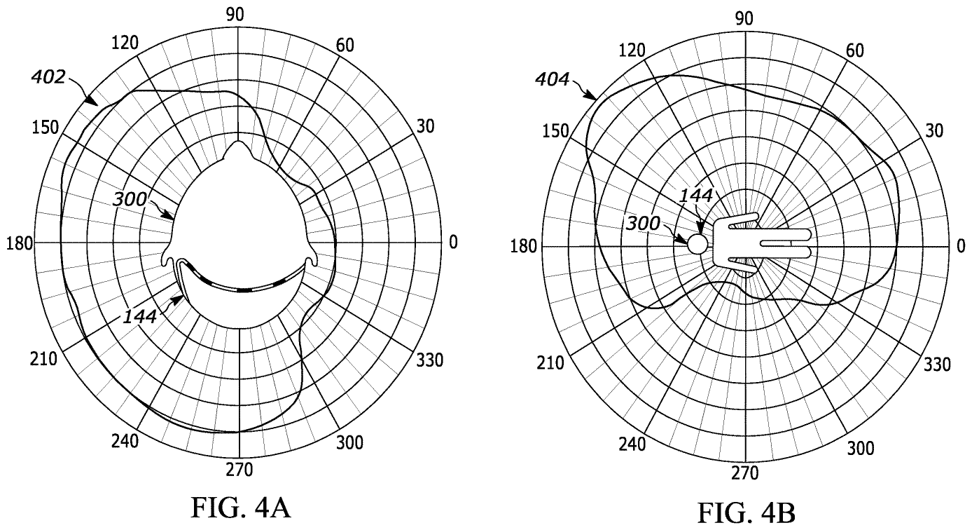

FIGS. 4A and 4B depict example radiation patterns for wireless communication for an implant device 300, as described above with regard to FIGS. 1-3I. In particular, FIG. 4A depicts a radiation pattern about an axis extending from the top of a hypothetical patient's head and FIG. 4B depicts a radiation pattern about an axis extending through the patient's abdomen (i.e., orthogonal to the axis of FIG. 4A). As illustrated in FIGS. 4A and 4B, the radiation pattern indicates that the wireless field is stronger on the side of the user including the local processing device 144 (and, as such, the transceiver 150 depicted in FIG. 1). The presence of organic materials between the local processing device 144 and the other side of the head (e.g., the skull, skin, brain, etc.) and/or water (e.g., in the human body) impedes the field and can contribute to weaker signal in transmitting data (or, conversely, higher transmission power requirements). As such, determining when to transmit data should not rely solely on distance between a sensor array of implant device 300 and an external computing device (e.g., processor device 104 of FIGS. 1 and 2), but should include other factors, such as a packet error rate (PER) indicative of the general strength of the field at the external computing device's location. The implant device 300 or an external computing device (e.g., processor device 104 as depicted in FIGS. 1 and 2) may therefore calculate a PER at various times to determine a preferred transmission time period for transmissions from the implant device 300 to the external computing device.

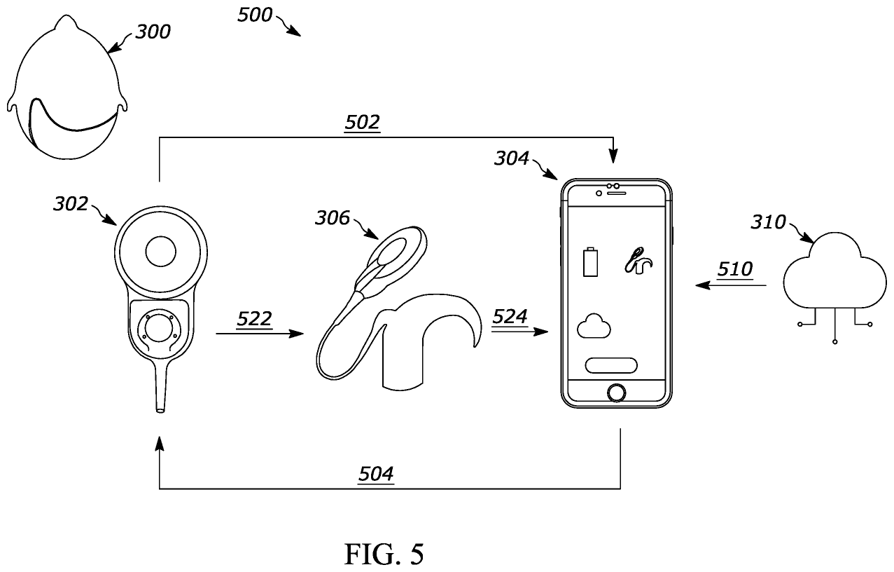

FIG. 5 depicts a system 500 for facilitating wireless communication between a sensor array 302 in an implant device 300 and an external computing device 304. Depending on the embodiment, the system 500 may additionally include an intermediate device 306 to facilitate communications between the sensor array 302 and the external computing device 304. Similarly, in some embodiments, the external computing device 304 is communicatively coupled with a cloud network 310 associated with one or more other computing devices and/or computing storage. It will be understood that, depending on the embodiment, the components of system 500 may include one or more components as described herein. For example, the implant device 300 including the sensor array 302 may be and/or include components of the system 100 described above with regard to FIGS. 1-3I (e.g., the sensor array 102 and components thereof). Similarly, the external computing device 304 may be and/or include the processor device 104 and/or a device with similar functionalities as described above with regard to FIGS. 1 and 2. Further, as described herein, the external computing device 304 may be or include a cell phone, wearable device (e.g., smart watch), mobile electronic device, computer, etc.

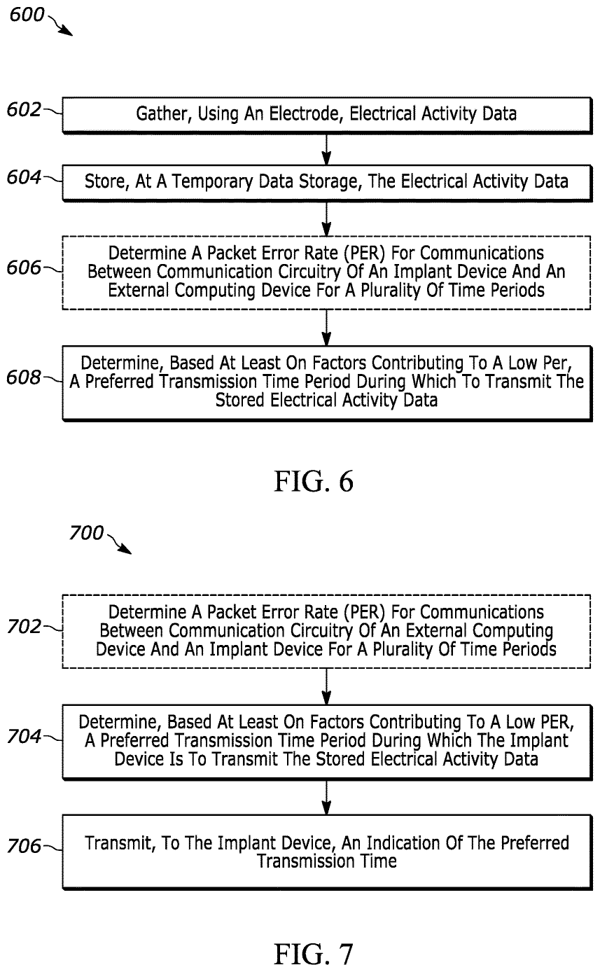

In some embodiments, the sensor array 302 transmits 502 data (e.g., EEG data gathered by one or more electrodes, accelerometer data, microphone data, etc.) directly to the external computing device 304 and receives 504 feedback from the external computing device 304. In further embodiments, the sensor array 302 determines a preferred transmission time period during which to transmit 502 the data to the external computing device 304, as described in more detail with regard to FIGS. 6 and 7 below. In other embodiments, the external computing device 304 determines the preferred transmission time period and transmits an indication of the preferred transmission time period to the sensor array 302, as described in more detail with regard to FIG. 7 below. Depending on the embodiment, the sensor array 302 may receive 504 feedback including a measured packet error rate (PER) (e.g., to be used in determining the preferred transmission time period), determined factors contributing to the measured PER, the determined preferred transmission time period, one or more limitations regarding the preferred transmission time period (e.g., only at night, not during working hours, only in particular locations, etc.), and/or any other such communications from the external computing device 304 to the sensor array 302 associated with the operations described herein.

In further embodiments, the system 500 includes an intermediate device 306 that acts as a base station and/or relay for communications between the sensor array 302 and the external computing device 304. In such embodiments, the sensor array 302 transmits 522 data to the intermediate device 306 (e.g., via Wi-Fi, Bluetooth, 5G, WLAN, RFID, and/or any other such technique), which in turn transmits 524 the data to the external computing device 304 (e.g., via Wi-Fi, Bluetooth, 5G, WLAN, RFID, and/or any other such technique). Depending on the embodiment, the intermediate device 306 may be, include, or function similarly to the processor device 104, and may therefore include communication circuitry similar to communication circuitry 256, a microprocessor similar to microprocessor 258, and/or a memory similar to memory 260. As such, the intermediate device 306 may temporarily store data received from the sensor array 302 to later transmit 524 to the external computing device 304. In other embodiments, the intermediate device 306 automatically transmits 524 any received data to the external computing device 304 upon receipt, thereby providing relay and/or amplification functionality. Depending on the embodiment, the sensor array 302 may determine to transmit to the intermediate device 304 when the preferred transmission time period is too far in the future (e.g., the sensor array 302 determines the preferred transmission time period is past a predetermined time threshold in the future, the memory will reach a predetermined threshold storage capacity before the time period, a priority event occurs, the subject or another user indicates an immediate transmission should occur, etc.).

Depending on the embodiment, the intermediate device 306 may be and/or include a wearable device to be worn by a user near the implant device 300 (e.g., worn behind and/or on the user's ear), for example, for recharging the battery of the implant device 300 or for communicating other information to or from the implant device 300. In other embodiments, the intermediate device 306 is a device for the user to hold elsewhere on the user's person (e.g., as a watch, phone, apparatus, etc.). In some embodiments, the intermediate device includes one or more sensors and gathers data related to the user's positioning, communication between the devices, etc. For example, the intermediate device 306 may determine a PER between the sensor array 302 and the intermediate device 306 and/or between the intermediate device 306 and then external computing device 304. Similarly, the intermediate device 306 may include one or more motion sensors (e.g., accelerometer, 3-axis gyroscope, magnetometer, etc.) to measure the positioning of the user. The intermediate device 306 may transmit such data to the sensor array 302 and/or external computing device 304 for use in determining a PER, preferred transmission time period, etc.

In some embodiments, the cloud network 310 may store one or more algorithms for characterizing a user's pattern of use, total storage available on the implant device 300, a PER between the sensor array 302 and the external computing device 304, a preferred transmission time period, etc. Similarly, the cloud network 310 may store one or more trained models (e.g., via machine learning and/or artificial intelligence techniques). The cloud network 310 may transmit 310 feedback to the external computing device 304, indicating a signal strength (e.g., via PER) of the wireless connection and/or other such metrics noted herein. In further embodiments, the cloud network 310 is additionally communicatively coupled to the sensor array 302 and/or the intermediate device 306. The cloud network 310 may receive an indication from a device of system 500 (or a computing device outside the system but communicatively coupled to a component of system 500) including an indication to generate a contact event at computing device 304 to cause a user to bring the external computing device 304 closer to the implant device 300, thereby reducing the PER. In still further embodiments, the sensor array 302 or the intermediate device 306 may provide an indication to the external computing device 304 to cause the external computing device 304 to generate the contact event. In yet still further embodiments, the external computing device 304 may determine to generate the contact event without prompting from the sensor array 302, intermediate device 306, or cloud network 310 (e.g., in response to data gathered or received by the external computing device 304).

In some such embodiments, the contact event includes a phone call, text message, application notification, sound, etc. The contact event may include an explanation (e.g., a message notifying the user that a download is transferring and to stay on the line and/or keep the phone near the user's head until a subsequent notification occurs). In some embodiments, the cloud network 310 and/or sensor array 302 may generate the contact event responsive to determining that the preferred transmission time period is too far in the future (e.g., the sensor array 302 determines the preferred transmission time period is past a predetermined time threshold in the future, the memory will reach a predetermined threshold storage capacity before the time period, a priority event occurs, the subject or another user indicates an immediate transmission should occur, etc.).

In further embodiments, the contact event may include the external computing device 304 and/or intermediate device 306. For example, the intermediate device 306 may notify a user (e.g., via an audio notification, a visual notification, a vibration, etc.) to guide the user in performing the contact event. In some examples, the intermediate device 306 displays a message to the user requesting that the user move the external computing device towards the sensor array 302. Similarly, the intermediate device 306 and/or the external computing device 304 may provide haptic, pulsatile, and/or tactile feedback and/or audio queues to guide the user to orient themselves (and, by extension, the sensor array 302) to better align with an improved PER for communications. In still further embodiments, the contact event may include haptic feedback (e.g., vibrations, buzzing, etc.) for the user in the sensor array 302, external computing device 304, and/or intermediate device 306 responsive to a determination (e.g., in response to data storage passing a predetermined threshold value) and to indicate to the user to move closer to the external computing device 304 and/or intermediate device 306 for data transfer.

FIG. 6 illustrates a method 600 in which an implant device (e.g., sensor array 102) gathers and wirelessly transmits data (e.g., EEG data, microphone data, accelerometer data, user reported data, etc.) at a determined preferred transmission time. Although the method 600 may utilize components of the system 100 (e.g., sensor array 102, processor device 104, and/or various components thereof), it will be understood that other components, devices, etc. according to FIGS. 1-5 may similarly perform the method 600.

At block 602, the implant device gathers activity data associated with a user. In particular, the implant device may gather data associated with the user's brain, such as EEG data via one or more electrodes (e.g., electrodes 110). Additionally or alternatively, the implant device may gather more general data associated with the user, such as microphone data, accelerometer data, user reported data, etc. For example, in some embodiments, the accelerometer, microphone, and/or other sensors may provide data indicating that a user is laying down and/or sitting for long periods of time (e.g., 1 hour, 6 hours, 8 hours, etc.).

At block 604, the implant device stores the gathered data temporarily at a data storage. In some embodiments, the implant device stores the gathered data for a predetermined period of time prior to deletion (e.g., hours, days, weeks, etc.). In further embodiments, the implant device stores the gathered data until the temporary data storage hits a predetermined data storage threshold, at which point the implant device automatically deletes some of the stored data (e.g., by oldest first, by non-priority data, by data size, etc.). In still further embodiments, the implant device stores the gathered data until transmitting the data to an external computing device (e.g., processor device 104), at which point the implant device deletes any data transmitted to the external computing device. In some such embodiments, the implant device may wait for a confirmation of receipt from the external computing device before deleting transmitted data. Depending on the embodiment, the implant device may similarly utilize other techniques for maintaining memory and/or a combination of such and/or the techniques as described above.

At block 606, the implant device may determine a packet error rate (PER) for communications between the implant device and an external computing device. As discussed in more detail above with regard to FIG. 1, the external computing device may be the processor device 104 or another computing device communicatively coupled to the processor device 104. In some embodiments in which the external computing device is a device coupled to the processor device 104, the processor device 104 may determine the PER in addition to or alternatively to the implant device. In further embodiments, the processor device 104 as the external computing device (and/or another computing device) may determine the PER, as described below with regard to FIG. 7.

In some embodiments, the implant device determines the PER by measuring one or more factors that contribute to the PER. For example, the sensor 102 may transmit one or more messages to the external computing device and determine the PER based on feedback from the device. Similarly, the implant device may receive and/or record data associated with factors that may influence the PER. For example, the implant device may automatically determine that one or more organic obstacles would cause an increased PER as the implant device is a subdermal implant. Similarly, the implant device may detect frequent electromagnetic interference, water, and/or the presence of a non-organic physical obstacle (e.g., a hat) and determine when the electromagnetic interference will be present using a machine learning and/or otherwise trained model. Alternatively or additionally, the external computing device may record measurements and/or data of factors that contribute to the PER and transmit the measurements and/or data to the implant device for the determination.

Depending on the embodiment, the implant device may determine the and/or factors contributing to the PER as associated with a plurality of time periods for the user. For example, the implant device may determine the PER every day, week, etc. at a predetermined number of time periods (e.g., early morning, noon, early evening, night, etc.). The implant device may then use the determined PER for the relevant time period to predict a PER for a particular corresponding time period. For example, the implant device may determine the PER every night at midnight for one week and may use the results to predict a PER for a future night at midnight. The implant device may automatically select one or more predetermined times for determining the PER, may receive preferred times from a user to determine the PER, etc.

In further embodiments, the implant device may correlate additional data (e.g., accelerometer data, microphone data, user-provided data, etc.) with a PER and determine that, for example, the PER is improved when the user is laying down for long periods of time (e.g., because the user has an external computing device such as a cell phone nearby while sleeping). As such, the processor device 104 may determine a PER for periods of time with similar accelerometer data and/or determine a preferred transmission time period based on such accelerometer, microphone, sensor, etc. data.

In still further embodiments, the implant device may measure the PER according to one or more details of a technical communication standard (e.g., the Bluetooth LE standard according to IEEE 802.15). In such embodiments, the implant device may perform test transmissions, gather data according to historical transmission data, and/or gather data from standard data transmissions according to the technical communication standard.

At block 608, the implant device determines a preferred transmission time period during which to transmit the stored activity data to the external computing device. Depending on the embodiment, the implant device may determine the preferred transmission time period in real-time and/or prior to the preferred transmission time period. For example, the implant device may determine the preferred transmission time as part of a scheduled event (e.g., the implant device determines that midnight is a preferred time period and schedules the data transmission to begin at midnight) or opportunistically (e.g., in response to a factor such as location, person position, cellphone near ear, memory (storage constraint), event-based, etc.). In further embodiments, the implant device determines the preferred transmission time period based on a static model or a dynamic model (e.g., a model that is pretrained compared to one that is trained using determinations).

In some embodiments, the implant device determines the preferred transmission time period based on factors contributing to a PER and/or the determined PER as described with regard to block 606 above. For example, the implant device may determine the preferred transmission time period according to a determined current PER (e.g., determining that a current PER is below a threshold value, whether predetermined or determined in real time), according to a determined historical PER (e.g., a historical PER based on time of day, location, user position, external device location, distance between external device and implant device, etc.), and/or a received PER (e.g., as received from an external computing device, intermediate device, smart device, etc.). As another example, the preferred transmission time period may rely exclusively on the factors that contribute to a low PER, such as a time of day, a position of a user, a position of the external device, a location of the user, a location of the external device, time since last data download, time to future predicted and/or scheduled data download, etc.

In some such embodiments, the external computing device provides feedback to the implant device regarding particular times, locations, power levels, etc. during which the implant device is to transmit the stored activity data. In such embodiments, the implant device further determines the preferred transmission time period based on the feedback. For example, the implant device may receive an indication to not transmit the stored activity data between the hours of 08:00-17:00, when the user is working. Depending on the embodiment, the external computing device may determine what time periods not to transmit the stored activity data on its own (e.g., responsive to consistently poor PERs, etc.) or in response to an indication from the user (e.g., do not disturb during a set time period).

In further embodiments, the implant device determines that a predetermined percentage of the temporary data storage is filled and begins opportunistically determining the preferred transmission time period responsive to such. For example, the implant device may begin determining the preferred transmission time period when the storage reaches 50% full, 70% full, 75% full, 80% full, etc. In some such embodiments, the implant device determines the preferred transmission time period at an earlier time and transmits at the first time period available after reaching the storage threshold as noted above. In further such embodiments, the implant device determines that the predetermined percentage of the temporary data storage is filled and automatically determines the preferred transmission time based on one or more factors that contribute to a relatively low PER within a particular time period. For example, after reaching 75% full, the implant device determines that the memory will fill within 6 hours. The implant device may then, depending on the embodiment, determine to transmit data to the external computing device (i) when one or more factors indicative of low PER are met, (ii) at a predicted future low PER, (iii) when a PER below a predetermined threshold is determined, (iv) responsive to forcing a contact event as described herein, (v) immediately (e.g., upon a determination that the PER will not be lower within the time period), or (vi) according to any other such element as described herein. In embodiments in which the implant device transmits data to an intermediate device (e.g., intermediate device 306 of FIG. 5), the implant device may transmit data based on whether the battery of the intermediate device is being charged by the intermediate device (e.g., as an inductive charging circuit that includes a transmitter in the intermediate device).

In further embodiments, the implant device calculates an estimated length of transmission based at least on the stored electrical activity data and determines the preferred transmission time period further based on the estimated length of transmission. For example, if the implant device determines that 05:00-05:05 is the preferred transmission time period, but the estimated length of transmission is 7 minutes, the implant device may determine a different preferred transmission time period. In alternate embodiments, the implant device transmits data until the end of the preferred transmission time period and sends the remainder of the data during another preferred transmission time period. Similarly, in some embodiments, the implant device determines that one or more events with increased priority have occurred (e.g., a seizure event) and flags the events in question. The implant device then determines a preferred transmission time period for the data associated with the event. In other embodiments, the implant device determines the preferred transmission time period as normal but transmits the prioritized data first rather than from oldest data to newest data.

In still further embodiments, the implant device may generate and/or transmit an indication to the external computing device to cause a contact event to temporarily decrease the PER. For example, the implant device may transmit an indication to the external computing device to cause a phone call so that the user raises the phone to an ear, allowing for close contact and transfer from the implant device to the phone (e.g., with decreased PER). In some embodiments, the implant device generates and/or transmits the indication in response to detecting one or more errors while attempting to transmit the stored data during the preferred transmission time period. In further embodiments, the implant device may generate and/or transmit the indication at another time during the preferred transmission time period (e.g., responsive to hitting a predetermined memory storage threshold, responsive to a predetermined number of days passing without transfer, responsive to a priority event occurring, etc.).

Depending on the embodiment, the preferred transmission time period may depend on any one or combination of a plurality of factors. For example, the system 100 may determine the preferred transmission time period based on any of: a determined presence of water, a determined presence of organic obstacles, a determined presence of non-organic obstacles, a determined location of the external computing device relative to the implant device, accelerometer data, microphone data, user-provided data, an expected time for a storage capacity threshold to be exceeded, a determination that a storage capacity threshold is exceeded, a presence of a priority event, etc. It will be understood that the foregoing factors are exemplary only and should not be construed as an exclusive list.

Depending on the embodiment, the implant device may determine the PER, factors contributing to a PER, and/or the preferred transmission time period according to one or more trained models (e.g., machine learning (ML) and/or artificial intelligence (AI) models). In some embodiments, an external device trains the model using depersonalized historical data and later transmits the model to the implant device. In further embodiments, one of the implant device or the external computing device trains the model using data personalized to the user (e.g., collected from the user) in conjunction with or separately from the depersonalized historical data. For example, the implant device may train an Al model to determine a PER using past measured PER score data, time data, location data, etc. As another example, the implant device may train an AI model to determine one or more factors that affect the PER (e.g., factors that have an impact on the PER of more than 1%, 5%, 10%, etc.). As yet another example, the implant device may train an AI model to determine the preferred transmission time period using historical PERs, factors contributing to a small PER, etc. As such, the AI model may predict a future PER and/or preferred transmission time period for a particular set of factors (e.g., time, location, distance from the external computing device, etc.). The AI model may further determine when a user is not compliant with alerts or predictions as to the preferred transmission time period (e.g., when the patient does not cooperate with or ignores indications to allow for data transfer). In further embodiments, the AI model interfaces with medication management systems and/or applications. Therefore, the AI model may determine that a preferred transmission time period intersects with a user entering drug intake data or other such data into a phone or other mobile device. In some embodiments, the trained model may determine whether the predicted data were accurate and may adjust the model accordingly. As such, the trained model may continually update predictions based on any accumulated data.

FIG. 7 illustrates a method 700 similar to method 600 in which an external computing device (e.g., processor device 104) determines a preferred transmission time period for an implant device (e.g., sensor array 102) to wirelessly transmit gathered data (e.g., EEG data, PPG data, microphone data, accelerometer data, user reported data, etc.). Although the method 700 may utilize components of the system 100 (e.g., sensor array 102, processor device 104, and/or various components thereof), it will be understood that, similar to the method 600, other components, devices, etc. according to FIGS. 1-5 may similarly perform the method 700.

At block 702, the external computing device may determine a PER for communications between the external computing device and the implant device, similar to block 606 described with regard to FIG. 6 above. In some embodiments, the external computing device determines the PER by measuring one or more factors that contribute to the PER. For example, the external computing device may receive one or more messages from the implant device and determine the PER based on feedback from the device. Similarly, the external computing device may receive and/or record data associated with factors that may influence the PER (e.g., from the implant device, from a cloud server, by one or more sensors of the external computing device, etc.). For example, the external computing device may automatically determine that one or more organic obstacles would cause an increased PER as the implant device is a subdermal implant. Similarly, the external computing device may detect and/or receive an indication (e.g., from the implant device, from another computing device, from the user, etc.) of frequent electromagnetic interference, water, and/or the presence of a non-organic physical obstacle (e.g., a hat) and determine when the electromagnetic interference will be present using a machine learning and/or otherwise trained model.

In some embodiments, the external computing device determines a location of the implant device by emitting a high frequency signal (e.g., emitting a tone at frequencies above human hearing). For example, during night hours when the user is expected to be sleeping, the external computing device may emit a high frequency tone to cause the implant device to transmit a message, emit a responsive signal, and/or otherwise reply to the external computing device to confirm a location and/or range (e.g., in conjunction with a ResMed device).

At block 704, the external computing device determines a preferred transmission time period during which the implant device is to transmit the stored electrical activity data, similar to block 608 described with regard to FIG. 6 above. As such, additional embodiments as described with regard to block 608 may similarly apply to block 704 to the extent one of skill in the art would recognize the external computing device as capable of performing equivalent functionality to the implant device described above.

At block 706, the external computing device transmits the indication of the preferred transmission time to the implant device. In some embodiments, the external computing device transmits the indication of the preferred transmission time responsive to determining the preferred transmission time period. In further embodiments, the external computing device transmits the indication of the preferred transmission time period responsive to receiving a request from the implant device and/or an indication that the implant device should transmit data (e.g., an indication that temporary data storage is above a predetermined threshold, an indication of data associated with a priority event, an indication from a user, etc.).

Depending on the embodiment, the implant device may attempt to transmit at the preferred transmission time even if the measured PER is lower than the expected PER. For example, the implant device may, after receiving an indication of the preferred transmission time, measure the PER shortly prior to or at the preferred transmission time. The implant device then, if the PER is above a predetermined threshold and/or higher than a predicted PER (e.g., more errors are occurring in a measured transmission), adaptively boost a signal (e.g., of a Bluetooth transmitter or other wireless transceiver) to attempt to transmit the data anyway. In further embodiments, the implant device transmits a message to the external computing device upon determining that the PER is high and requests a new preferred transmission time.

In some embodiments, the external computing device includes an indication that the implant device must or should transmit the data at an indicated time, or the implant device determines that the data must or should be transmitted at an indicated time or in response to a determination (e.g., when memory is almost full or urgent data is gathered, as described above). As such, the implant device adaptively boosts the signal to transmit such data. Therefore, the implant device selectively increases signal power and subsequent power consumption when necessary while maintaining an overall decreased power consumption.

In some embodiments, the external computing device communicates with the implant device via an intermediate device (e.g., intermediate device 302 as described above with regard to FIG. 5) rather than directly. For example, a wearable relay device may act as a base station for communications between the implant device and the external computing device. Depending on the embodiment, the implant device may transmit data to the intermediate device and/or the external computing device may transmit an indication to the intermediate device for the implant device to transmit data responsive to the various factors as described above with regard to FIGS. 6 and/or 7. For example, the implant device may determine that the temporary memory will fill before a preferred transmission time period, and may transmit at least some of the stored data to the intermediate device for storage and/or to transmit to the external computing device.

The following list of aspects reflects a variety of the embodiments explicitly contemplated by the present disclosure. Those of ordinary skill in the art will readily appreciate that the aspects below are neither limiting of the embodiments disclosed herein, nor exhaustive of all of the embodiments conceivable from the disclosure above, but are instead meant to be exemplary in nature.

Aspect 1. An implant device configured to be implanted in a human and to wirelessly transmit, to an external computing device at determined times, gathered data, the implant device comprising: a plurality of electrodes configured to gather electrical activity data associated with a brain of a user; communication circuitry configured to wirelessly communicate with the external computing device; a processing device; and a data storage device configured to temporarily store data and including a computer-readable media storing machine readable instructions that, when executed, cause the processing device to: gather, using the electrode, the electrical activity data; store, at the data storage device, the electrical activity data; and determine, based at least on one or more factors contributing to a low packet error rate (PER) for communications between the implant device and the external computing device for a plurality of time periods, a preferred transmission time period of the plurality of time periods during which to transmit the stored electrical activity data to the external computing device.

Aspect 2. The implant device of aspect 1, wherein the computer-readable media further stores instructions that, when executed, cause the processing device to: determine a packet error rate (PER) for communications between the communication circuitry and the external computing device for a plurality of time periods; wherein the preferred transmission time period is based on the PER.

Aspect 3. The implant device of either of aspect 1 or 2, further comprising an accelerometer, wherein the determining the preferred transmission time period includes: measuring, using the accelerometer, a movement value for the implant device for the plurality of time periods; and calculating, based at least on the measured movement value, the preferred transmission time period.

Aspect 4. The implant device of any one of aspects 1 to 3, wherein the computer-readable media further stores instructions that, when executed, cause the processing device to: receive, from the external device, feedback associated with the communications; wherein the determining the preferred transmission time period is further based at least on the feedback associated with the communications.

Aspect 5. The implant device of any one of aspects 1 to 4, wherein the determining the preferred transmission time period includes: analyzing the one or more factors for the plurality of time periods using a trained machine learning algorithm to generate a PER factor analysis; and determining the preferred transmission time period based at least on the PER factor analysis.

Aspect 6. The implant device of any one of aspects 1 to 4, wherein the determining the preferred transmission time period includes: transmitting data associated with the one or more factors for the plurality of time periods to an external analysis device; receiving a PER factor analysis generated by a trained machine learning algorithm from the external analysis device; and determining the preferred transmission time period based at least on the PER factor analysis.

Aspect 7. The implant device of aspect 6, wherein the external computing device includes the external analysis device.

Aspect 8. The implant device of any one of aspects 1 to 7, wherein the PER is based on at least one of: (i) electromagnetic interference, (ii) a presence of one or more non-organic physical obstacles, (iii) a presence of one or more organic obstacles, or (iv) a location of the external computing device relative to the communications circuitry.

Aspect 9. The implant device of any one of aspects 1 to 8, wherein the computer-readable media further stores instructions that, when executed, cause the processing device to: determine that a predetermined percentage of the temporary data storage is filled; wherein the determining the preferred transmission time period is responsive to and further based at least on the determining that the predetermined percentage of the temporary data storage is filled.

Aspect 10. The implant device of aspect 9, wherein the computer-readable media further stores instructions that, when executed, cause the processing device to: transmit a signal to the external computing device to cause the external computing device to alert the user that the predetermined percentage of the temporary data storage is filled.

Aspect 11. The implant device of aspect 10, wherein alerting the user includes providing haptic feedback to the user to indicate that the predetermined percentage of the temporary data storage is filled.

Aspect 12. The implant device of any one of aspects 1 to 11, wherein the computer-readable media further stores instructions that, when executed, cause the processing device to: calculate an estimated length of transmission based at least on the stored electrical activity data; and wherein the determining the preferred transmission time period is further based at least on the estimated length of transmission.

Aspect 13. The implant device of any one of aspects 1 to 12, wherein the computer-readable media further stores instructions that, when executed, cause the processing device to: determine that an event with an increased priority has occurred; wherein the preferred transmission time period is determined for data associated with the event.

Aspect 14. The implant device of any one of aspects 1 to 13, wherein the computer-readable media further stores instructions that, when executed, cause the processing device to: transmit an indication to the external computing device to cause a contact event to temporarily decrease a current PER for communications between the implant device and the external computing device.

Aspect 15. The implant device of aspect 14, wherein the external computing device is configured to perform phone call functionality and the contact event to temporarily decrease the current PER is a phone call to cause the user to move the external computing device closer to the implant device.

Aspect 16. The implant device of either of aspect 14 or 15, wherein the transmitting the indication occurs during the preferred transmission time period.

Aspect 17. The implant device of either of aspect 14 or 15, wherein the transmitting the indication is responsive to detecting one or more errors while attempting to transmit during the preferred transmission time period.

Aspect 18. The implant device of any one of aspects 14 to 17, wherein the computer-readable media further stores instructions that, when executed, cause the processing device to: determine, using the machine learning model, that a user does not respond to the indication.

Aspect 19. The implant device of any one of aspects 14 to 18, wherein the indication includes at least one of: (i) a signal to cause the external computing device to emit an audio cue, (ii) a signal to cause the external computing device to emit a visual cue, (iii) a signal to cause the external computing device to vibrate, (iv) a signal to cause the external computing device to guide a user in performing the contact event, or (v) a signal to cause the external computing device to guide a user in orienting the external computing device.

Aspect 20. The implant device of any one of aspects 14 to 19, wherein the indication includes instructions for guiding a user to a preferred communication location.

Aspect 21. The implant device of any one of the preceding aspects, wherein the computer-readable media further stores instructions that, when executed, cause the processing device to: responsive to determining that the PER satisfies a predetermined threshold, increase a power supplied to the communication circuitry.

Aspect 22. The implant device of any one aspects 1 to 20, wherein the computer-readable media further stores instructions that, when executed, cause the processing device to: responsive to determining that the PER satisfies a predetermined threshold at the preferred transmission time, determine a second preferred transmission time.

Aspect 23. A method for wirelessly transmitting gathered data from an implant device configured to be implanted in a human to an external computing device at determined times, the method comprising: gathering, by one or more processors, electrical activity data associated with a brain of a user via a plurality of electrodes of the implant device; storing, by the one or more processors and at a data storage device of the implant device configured to temporarily store data, the electrical activity data; and determining, by the one or more processors and based at least on one or more factors contributing to a low packet error rate (PER) for communications between the implant device and the external computing device for a plurality of time periods, a preferred transmission time period of the plurality of time periods during which to transmit the stored electrical activity data to the external computing device.

Aspect 24. The method of aspect 23, further comprising: determining a packet error rate (PER) for communications between the communication circuitry and the external computing device for a plurality of time periods; wherein the preferred transmission time period is based on the PER.

Aspect 25. The method of either of aspect 23 or 24, wherein the determining the preferred transmission time period includes: measuring, using an accelerometer, a movement value for the implant device for the plurality of time periods; and calculating, based at least on the measured movement value, the preferred transmission time period.

Aspect 26. The method of any one of aspects 23 to 25, further comprising: receiving, at the implant device and from the external device, feedback associated with the communications; wherein the determining the preferred transmission time period is further based at least on the feedback associated with the communications.

Aspect 27. The method of any one of aspects 23 to 26, wherein the determining the preferred transmission time period includes: analyzing, at the implant device, the one or more factors for the plurality of time periods using a trained machine learning algorithm to generate a PER factor analysis; and determining the preferred transmission time period based at least on the PER factor analysis.

Aspect 28. The method of any one of aspects 23 to 26, wherein the determining the preferred transmission time period includes: transmitting data associated with the one or more factors for the plurality of time periods to an external analysis device; receiving, at the implant device and from the external analysis device, a PER factor analysis generated by a trained machine learning algorithm; and determining the preferred transmission time period based at least on the PER factor analysis.

Aspect 29. The method of aspect 28, wherein the external computing device includes the external analysis device.

Aspect 30. The method of any one of aspects 23 to 29, wherein the external computing device is a wearable computing device configured to receive the electrical activity data from the implant device and transmit the electrical activity data to a data collection device.

Aspect 31. The method of any one of aspects 23 to 30, further comprising: determining that a predetermined percentage of the temporary data storage is filled; wherein the determining the preferred transmission time period is responsive to and further based at least on the determining that the predetermined percentage of the temporary data storage is filled.

Aspect 32. The method of aspect 31, further comprising: transmitting a signal to the external computing device to cause the external computing device to alert the user that the predetermined percentage of the temporary data storage is filled.

Aspect 33. The method of aspect 32, wherein alerting the user includes providing haptic feedback to the user to indicate that the predetermined percentage of the temporary data storage is filled.

Aspect 34. The method of any one of aspects 23 to 33, further comprising: calculating an estimated length of transmission based at least on the stored electrical activity data; and wherein the determining the preferred transmission time period is further based at least on the estimated length of transmission.

Aspect 35. The method of any one of aspects 23 to 34, further comprising: determining that an event with an increased priority has occurred; wherein the preferred transmission time period is determined for data associated with the event.

Aspect 36. The method of any one of aspects 23 to 35, further comprising: transmitting, from the implant device to the external computing device, an indication to cause a contact event to temporarily decrease the current PER for communications between the implant device and the external computing device.

Aspect 37. The method of aspect 36, wherein the external computing device is configured to perform phone call functionality and the contact event to temporarily decrease the PER is a phone call to cause the user to move the external computing device closer to the implant device.

Aspect 38. The method of either of aspects 36 or 37, wherein the transmitting the indication occurs during the preferred transmission time period.

Aspect 39. The method of either of aspects 36 or 37, wherein the transmitting the indication is responsive to detecting one or more errors while attempting to transmit during the preferred transmission time period.

Aspect 40. The method of any one of aspects 36 to 39, further comprising: determine, using the machine learning model, that a user does not respond to the indication.

Aspect 41. The method of any one of aspects 36 to 40, wherein the indication includes at least one of: (i) a signal to cause the external computing device to emit an audio cue, (ii) a signal to cause the external computing device to emit a visual cue, (iii) a signal to cause the external computing device to vibrate, (iv) a signal to cause the external computing device to guide a user in performing the contact event, or (v) a signal to cause the external computing device to guide a user in orienting the external computing device.

Aspect 42. The method of any one of aspects 36 to 41, wherein the indication includes instructions for guiding a user to a preferred communication location.

Aspect 43. The method of any one of aspects 23 to 42, further comprising: responsive to determining that the PER satisfies a predetermined threshold, increasing a power supplied to the communication circuitry.

Aspect 44. The method of any one of aspects 23 to 42, further comprising: responsive to determining that the PER satisfies a predetermined threshold at the preferred transmission time, determining a second preferred transmission time.

Aspect 45. A computing device configured to communicate with and wirelessly receive data from an implant device implanted in a human at determined times, the computing device comprising: communication circuitry configured to wirelessly communicate with an implant device; a processing device; and a computer-readable media storing machine readable instructions that, when executed, cause the processing device to: determine, based at least on one or more factors contributing to a low packet error rate (PER) for communications between the implant device and the external computing device for a plurality of time periods, a preferred transmission time period of the plurality of time periods during which the implant device is to transmit electrical activity data associated with a brain of a user to the computing device; and transmit an indication of the preferred transmission time period.

Aspect 46. The computing device of aspect 45, wherein the computer-readable media further stores instructions that, when executed, cause the processing device to: determine a packet error rate (PER) for communications between the communication circuitry and the external computing device for a plurality of time periods; wherein the preferred transmission time period is based on the PER.

Aspect 47. The computing device of either of aspects 45 or 47, wherein the determining the preferred transmission time period includes: receiving a movement value for the implant device for the plurality of time periods; and calculating, based at least on the measured movement value, the PER.

Aspect 48. The computing device of any one of aspects 45 to 47, wherein the computer-readable media further stores instructions that, when executed, cause the processing device to: receive, from a cloud network, feedback associated with the communications; wherein the determining the preferred transmission time period is further based at least on the feedback associated with the communications.

Aspect 49. The computing device of any one of aspects 45 to 48, wherein the determining the preferred transmission time period includes: analyzing the one or more factors for the plurality of time periods using a trained machine learning algorithm to generate a PER factor analysis; and determining the preferred transmission time period based at least on the PER factor analysis.

Aspect 50. The computing device of any one of aspects 45 to 49, wherein the transmitting is to a wearable computing device configured to facilitate communications between the implant device and the computing device.

Aspect 51. The computing device of any one of aspects 45 to 50, wherein the computer-readable media further stores instructions that, when executed, cause the processing device to: determine that a predetermined percentage of the temporary data storage is filled; wherein the determining the preferred transmission time period is responsive to and further based at least on the determining that the predetermined percentage of the temporary data storage is filled.

Aspect 52. The computing device of aspect 51, wherein the computer-readable media further stores instructions that, when executed, cause the processing device to: alert the user that the predetermined percentage of the temporary data storage is filled.

Aspect 53. The method of aspect 52, wherein alerting the user includes providing haptic feedback to the user to indicate that the predetermined percentage of the temporary data storage is filled.

Aspect 54. The computing device of any one of aspects 45 to 53, wherein the computer-readable media further stores instructions that, when executed, cause the processing device to: determine that an event with an increased priority has occurred; wherein the preferred transmission time period is determined for data associated with the event.

Aspect 55. The computing device of any one of aspects 45 to 54, wherein the computer-readable media further stores instructions that, when executed, cause the processing device to: receive an indication that causes a contact event to temporarily decrease a current PER for communications between the implant device and the external computing device.

Aspect 56. The computing device of aspect 55, wherein the computing device is configured to perform phone call functionality and the contact event to temporarily decrease the current PER is a phone call to cause the user to move the computing device closer to the implant device.

Aspect 57. The computing device of either of aspect 55 or 56, wherein the receiving the indication occurs during the preferred transmission time period.

Aspect 58. The computing device of either of aspect 55 or 56, wherein the receiving the indication is responsive to detecting one or more errors while receiving the electrical activity data during the preferred transmission time period.

Aspect 59. The computing device of any one of aspects 55 to 58, further comprising: determine, using the machine learning model, that a user does not respond to the indication.

Aspect 60. The computing device of any one of aspects 55 to 59, wherein the indication includes at least one of: (i) a signal to cause the external computing device to emit an audio cue, (ii) a signal to cause the external computing device to emit a visual cue, (iii) a signal to cause the external computing device to vibrate, (iv) a signal to cause the external computing device to guide a user in performing the contact event, or (v) a signal to cause the external computing device to guide a user in orienting the external computing device.

Aspect 61. The computing device of any one of aspects 55 to 60, wherein the indication includes instructions for guiding a user to a preferred communication location.

Aspect 62. The computing device of any one of aspects 55 to 61, wherein the computer-readable media further stores instructions that, when executed, cause the processing device to: determine that the user has met a predetermined data transfer threshold; and provide a reward to the user based on the predetermined data transfer threshold.

Aspect 63. The computing device of any one of aspects 45 to 62, wherein the computer-readable media further stores instructions that, when executed, cause the processing device to: responsive to determining that the PER satisfies a predetermined threshold, transmit an indication to the implant device increase a power supplied to the communication circuitry.

Aspect 64. The implant device of any one of aspects 45 to 62, wherein the computer-readable media further stores instructions that, when executed, cause the processing device to: responsive to determining that the PER satisfies a predetermined threshold at the preferred transmission time, determine a second preferred transmission time.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this disclosure is referred to in this disclosure in a manner consistent with a single meaning, that is done for sake of clarity only so as to not confuse the reader, and it is not intended that such claim term be limited, by implication or otherwise, to that single meaning.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still cooperate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also may include the plural unless it is obvious that it is meant otherwise.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

This detailed description is to be construed as examples and does not describe every possible embodiment, as describing every possible embodiment would be impractical, if not impossible. One could implement numerous alternate embodiments, using either current technology or technology developed after the filing date of this application.

Upon reading this disclosure, those of skill in the art will appreciate still additional alternative structural and functional designs for evaluation properties, through the principles disclosed herein. Therefore, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

1. An implant device configured to be implanted in a human and to wirelessly transmit, to an external computing device at determined times, gathered data, the implant device comprising:
a plurality of electrodes configured to gather electrical activity data associated with a brain of a user;
communication circuitry configured to wirelessly communicate with the external computing device;
a processing device; and
a data storage device configured to temporarily store data and including a computer-readable media storing machine readable instructions that, when executed, cause the processing device to:
gather, using the electrode, the electrical activity data;
store, at the data storage device, the electrical activity data; and
determine, based at least on one or more factors contributing to a low packet error rate (PER) for communications between the implant device and the external computing device for a plurality of time periods, a preferred transmission time period of the plurality of time periods during which to transmit the stored electrical activity data to the external computing device.

2. The implant device of claim 1, wherein the computer-readable media further stores instructions that, when executed, cause the processing device to:
determine a packet error rate (PER) for communications between the communication circuitry and the external computing device for a plurality of time periods;
wherein the preferred transmission time period is based on the PER.

3. The implant device of claim 1, further comprising an accelerometer, wherein the determining the preferred transmission time period includes:
measuring, using the accelerometer, a movement value for the implant device for the plurality of time periods; and
calculating, based at least on the measured movement value, the preferred transmission time period.

4. The implant device of claim 1, wherein the computer-readable media further stores instructions that, when executed, cause the processing device to:
receive, from the external device, feedback associated with the communications;
wherein the determining the preferred transmission time period is further based at least on the feedback associated with the communications.

5. The implant device of claim 1, wherein the determining the preferred transmission time period includes:
analyzing the one or more factors for the plurality of time periods using a trained machine learning algorithm to generate a PER factor analysis; and
determining the preferred transmission time period based at least on the PER factor analysis.

6. The implant device of claim 1, wherein the determining the preferred transmission time period includes:
transmitting data associated with the one or more factors for the plurality of time periods to an external analysis device;
receiving a PER factor analysis generated by a trained machine learning algorithm from the external analysis device; and
determining the preferred transmission time period based at least on the PER factor analysis.

7. The implant device of claim 1, wherein the external computing device is a wearable computing device configured to receive the electrical activity data from the implant device and transmit the electrical activity data to a data collection device.

8. The implant device of claim 1, wherein the computer-readable media further stores instructions that, when executed, cause the processing device to:
determine that a predetermined percentage of the temporary data storage is filled;
wherein the determining the preferred transmission time period is responsive to and further based at least on the determining that the predetermined percentage of the temporary data storage is filled.

9. The implant device of claim 1, wherein the computer-readable media further stores instructions that, when executed, cause the processing device to:
calculate an estimated length of transmission based at least on the stored electrical activity data; and
wherein the determining the preferred transmission time period is further based at least on the estimated length of transmission.

10. The implant device of claim 1, wherein the computer-readable media further stores instructions that, when executed, cause the processing device to:
determine that an event with an increased priority has occurred;
wherein the preferred transmission time period is determined for data associated with the event.

11. The implant device of claim 1, wherein the computer-readable media further stores instructions that, when executed, cause the processing device to:
transmit an indication to the external computing device to cause a contact event to temporarily decrease a current PER for communications between the implant device and the external computing device.

12. A method for wirelessly transmitting gathered data from an implant device configured to be implanted in a human to an external computing device at determined times, the method comprising:
gathering, by one or more processors, electrical activity data associated with a brain of a user via a plurality of electrodes of the implant device;
storing, by the one or more processors and at a data storage device of the implant device configured to temporarily store data, the electrical activity data; and
determining, by the one or more processors and based at least on one or more factors contributing to a low packet error rate (PER) for communications between the implant device and the external computing device for a plurality of time periods, a preferred transmission time period of the plurality of time periods during which to transmit the stored electrical activity data to the external computing device.

13. The method of claim 16, further comprising:
determining a packet error rate (PER) for communications between the communication circuitry and the external computing device for a plurality of time periods;
wherein the preferred transmission time period is based on the PER.

14. The method of claim 12, wherein the determining the preferred transmission time period includes:
measuring, using an accelerometer, a movement value for the implant device for the plurality of time periods; and
calculating, based at least on the measured movement value, the preferred transmission time period.

15. The method of claim 12, wherein the determining the preferred transmission time period includes:
analyzing, at the implant device, the one or more factors for the plurality of time periods using a trained machine learning algorithm to generate a PER factor analysis; and
determining the preferred transmission time period based at least on the PER factor analysis.
